(19) United States
(12) Patent Application Publication
(10) Pub. No.: US 2021/0077274 A1
(43) Pub. Date: Mar. 18, 2021

(54) Expandable Spinal Interbody Implant

(71) Applicant: FBC Device ApS, Risskov (DK)

(72) Inventor: Bruce H. Robie, North Andover, MA (US)

(73) Assignee: FBC Device ApS, Risskov (DK)

(21) Appl. No.: 17/015,656

(22) Filed: Sep. 9, 2020

Related U.S. Application Data
(60) Provisional application No. 62/900,765, filed on Sep. 16, 2019.

(51) Int. Cl. A61F 2/44

(52) U.S. Cl. CPC A61F 2/4425; A61F 2002/30507; A61F 2/447

(57) ABSTRACT

An expandable spinal interbody implant is provided that includes first/second components that are movable between collapsed and expanded states. Spaced apart surfaces with defined radii are associated with one of the components and cooperate with slots defined in the other of the components. A locking mechanism, e.g., a locking screw, may secure the components in a desired relative orientation.

### CROSS-REFERENCE TO RELATED APPLICATION(S)

The present application claims priority benefit to a US provisional patent application entitled “Expandable Spinal Interbody Implant” which was filed on Sep. 16, 2019, and assigned Ser. No. 62/900,765. The entire content of the foregoing provisional application is incorporated herein by reference.

The present application is related in subject matter to Applicant's previously filed patent filings: (i) US Publication No. 2018/0325561 to Robie et al. entitled “Angulating Bone Plate,” (ii) U.S. Pat. No. 8,007,536 to Christensen entitled “Disc Implant,” (iii) U.S. Pat. No. 8,795,372 to Christensen entitled “Disc Implant,” and (iv) U.S. Pat. No. 10,357,375 to Christensen entitled “Method of Spinal Treatment.” The content of each of the foregoing patent filings is incorporated herein by reference.

### BACKGROUND

### Technical Field

The present disclosure is directed to an expandable spinal interbody implant that includes first/second components that are movable between collapsed and expanded states. Spaced apart surfaces with defined radii are associated with one of the components and cooperate with slots defined in the other of the components. A locking mechanism, e.g., a locking screw secures the components in a desired relative orientation.

### Background of Disclosure

Spinal plates are commonly used and there are many versions in the prior art. Prior art spinal plates generally consist of one or more structural elements that are connected to each of the vertebral bodies adjacent to the level(s) to be fused via screws passing through holes in the structural elements and into the vertebral bodies. Some type of locking mechanism is generally provided to prevent or resist screw migration back through the structural elements. Spinal plates can be used for fusions throughout the spine.

In some instances, there may be a benefit to adjust the lordosis of the segment. One way to achieve that adjustment is via an adjustable interbody fusion device, such as that described by U.S. Pat. No. 8,007,536 to Christensen, previously incorporated by reference. When adjusting the lordosis of the interbody device, there may also be a benefit to have a similar adjustment in the spinal plates.

As noted above, the present disclosure is directed to an expandable spinal interbody implant that includes first/second components that are movable between collapsed and expanded states. Spaced apart surfaces with defined radii are associated with one of the components and cooperate with slots defined in the other of the components. A locking mechanism, e.g., a locking screw secures the components in a desired relative orientation.

### SUMMARY

The present disclosure provides an advantageous expandable interbody implant. The implant includes first and second components that are repositionable relative to each other, and may be fixed in a desired expanded configuration, e.g., by tightening a set screw.

In exemplary embodiments, the disclosed expandable interbody implant includes a first component defining an upper surface, and a second component movable relative to the first component between a collapsed state and an expanded state. The first component includes at least one upwardly extending sheet that projects from the upper surface. In exemplary embodiments, a pair of upwardly extending, spaced sheets are provided. The second component defines at least one mating slot configured and dimensioned to receive the at least one upwardly extending sheet of the first component. In embodiments where two upwardly extending sheets are provided on the first component, a corresponding pair of mating slots are defined by the second component.

The at least one upwardly extending sheet is radiused relative to the upper surface and the at least one mating slot defines an internal radius. The radius of the at least one upwardly extending sheet and internal radius of the at least one mating slot are selected to permit relative movement between the first component and the second component when the at least one upwardly extending sheet travels relative to the at least one mating slot.

A locking mechanism may be associated with the expandable interbody implant, e.g., a locking screw, to releasably secure the expandable interbody implant in a desired orientation, e.g., a collapsed orientation or an expanded orientation.

The present disclosure also provides methods for deployment of the disclosed expandable interbody implant and, optionally, fixation at a desired orientation.

Additional features, functions and benefits of the disclosed expandable interbody implant are set forth in the detailed description which follows, particularly when read in conjunction with the appended figures.

### BRIEF DESCRIPTION OF DRAWINGS

To assist those of skill in the art in making and using the disclosed implant, reference is made to the accompanying figures, wherein:

FIG. 1 is an oblique view of an exemplary embodiment in a collapsed position, according to the present disclosure;

FIG. 2 is an oblique view of an exemplary embodiment in an expanded position;

FIG. 3 is an oblique view of the bottom component;

FIG. 4 is a side view of the bottom component, along line A in FIG. 3;

FIG. 5 is a top view of the bottom component;

FIG. 6 is an oblique view of the top component;

FIG. 7 is an oblique view of the top component, looking up from below;

FIG. 8 is a side view of the top component, looking at the trailing end;

FIG. 9 is an oblique view of the top component, looking up from below;

FIG. 10 is a top view of the top component;

FIG. 11 is a section view of the top component, along line B-B in FIG. 10;

FIG. 12 is an oblique view of an alternative embodiment in a collapsed position;

FIG. 13 is an oblique view of the bottom component of the embodiment shown in FIG. 12;

FIG. 14 is a side view of the bottom component, along line C in FIG. 13:

FIG. 15 is an oblique view of the top component of the embodiment shown in FIG. 12;

FIG. 16 is an oblique view of the top component;

FIG. 17 is a top view of the top component; and

FIG. 18 is a section view of the top component, along line D-D in FIG. 17.

### DESCRIPTION OF EXEMPLARY EMBODIMENT(S)

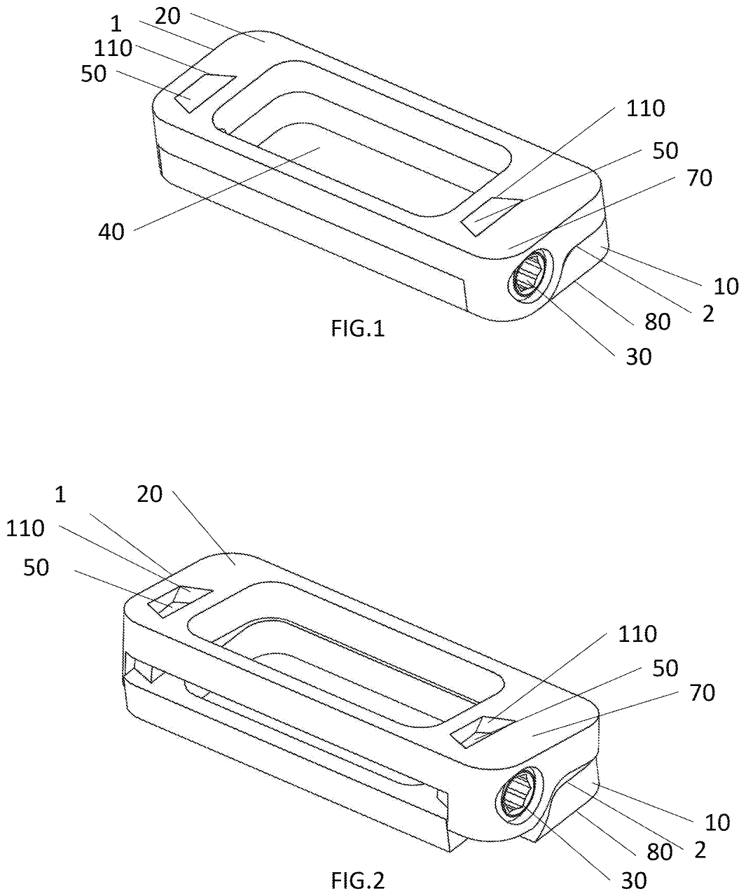

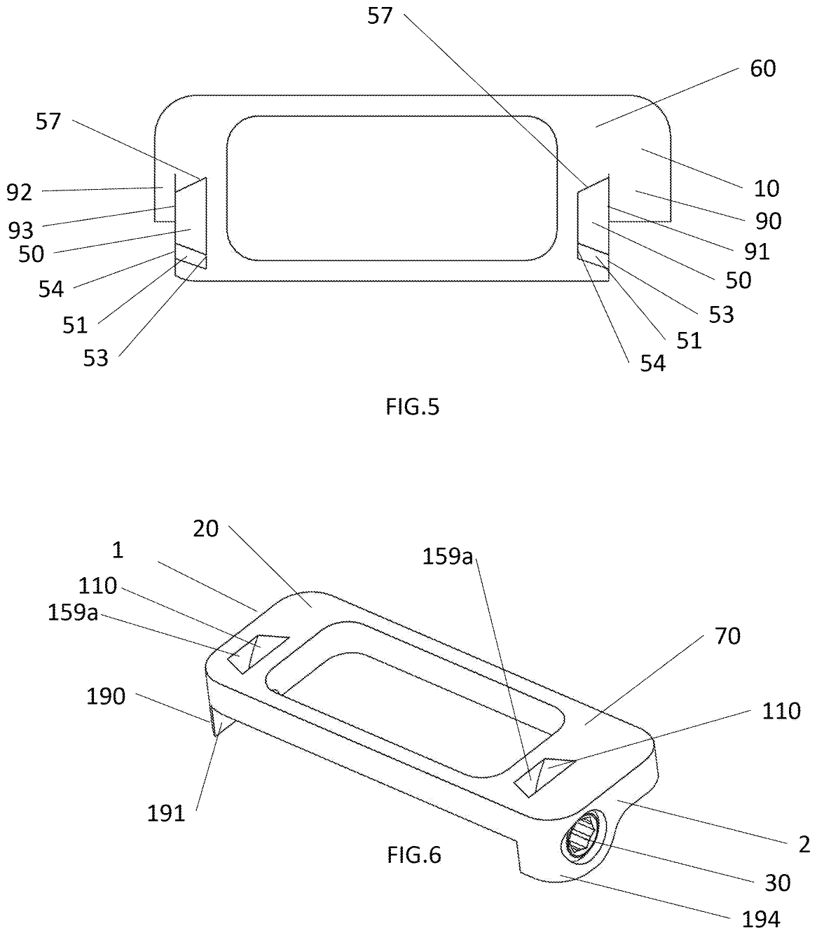

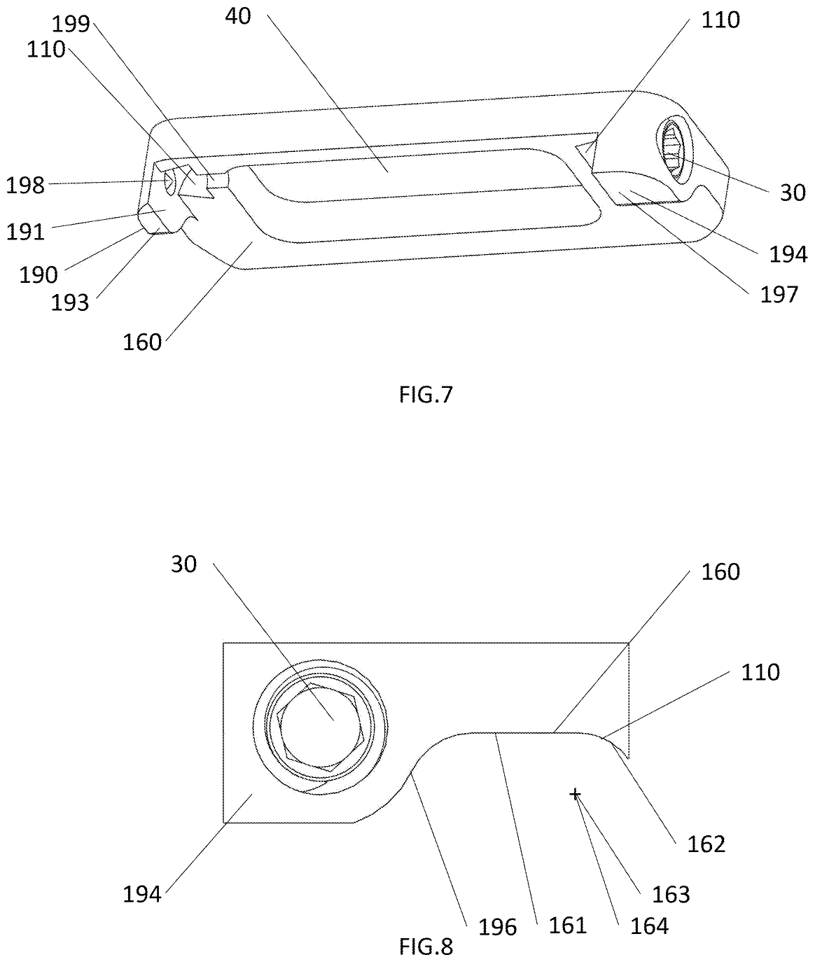

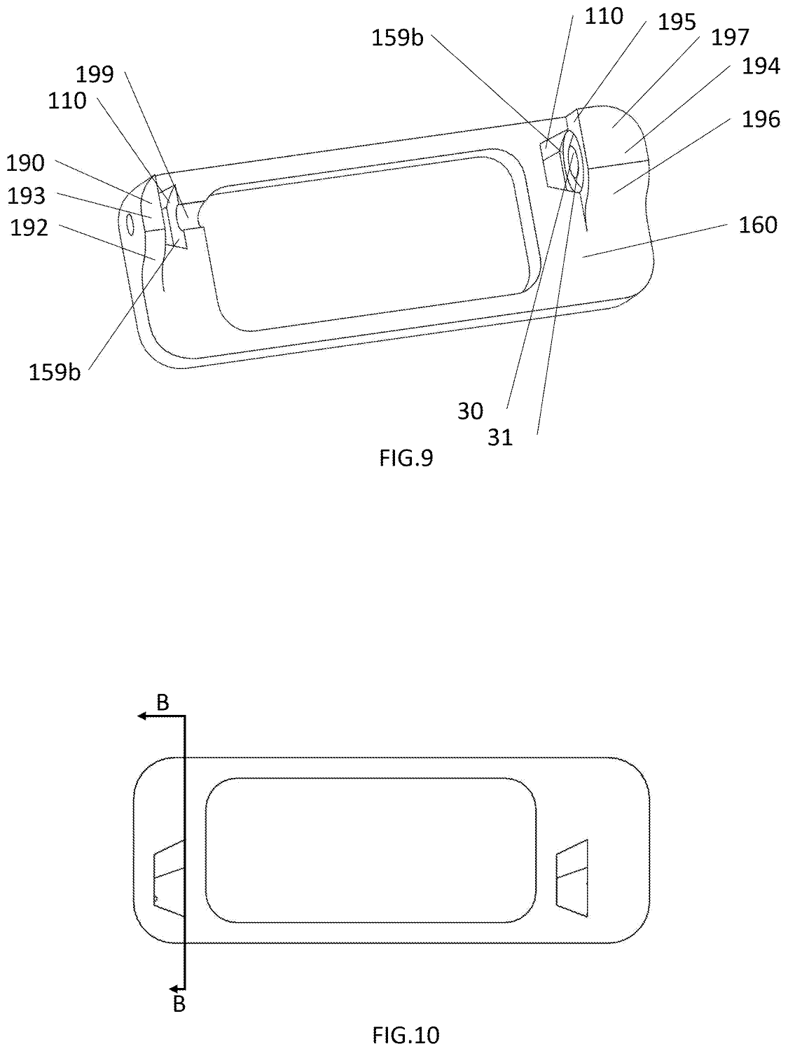

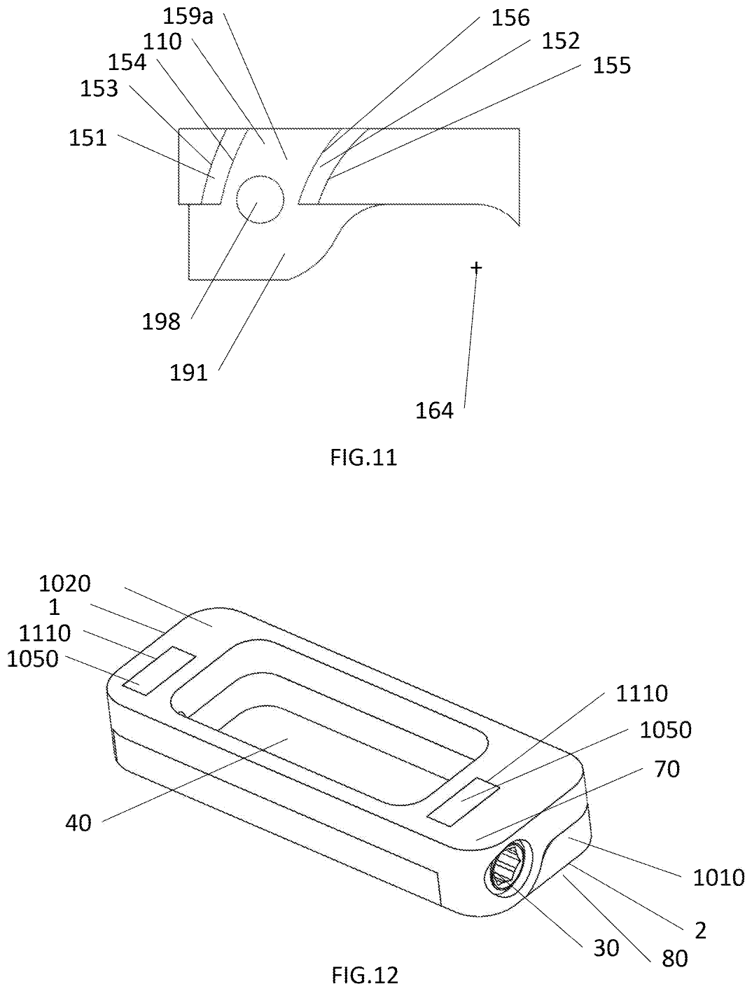

With reference to FIGS. 1-11, an exemplary implant is depicted schematically. With initial reference to FIGS. 1-2, oblique views are shown. The embodiment is composed of a bottom component 10, assembled with a top component 20, with a locking screw 30 threaded into the top component. Both top and bottom components include a cavity 40 for holding bone graft materials. The outer profiles and the cavity 40 are generally or substantially matched between the top and bottom components. There is a leading end 1 and a trailing end 2 of both components. There is an upper surface 70 and a lower surface 80. As shown in FIG. 1, these two surfaces are generally parallel. Alternatively, there could be an angle defined between the two surfaces in the collapsed state. FIG. 2 shows the exemplary implant in its expanded state. In the expanded state, the angle between the upper surface 70 and lower surface 80 has changed from the collapsed state, due to movement of the top component relative to the bottom component. The upper and lower surfaces may be flat (as shown), or curved in one or more planes. Further, the shape of the upper surface (e.g., flat, curved, irregular) may be different than the lower surface.

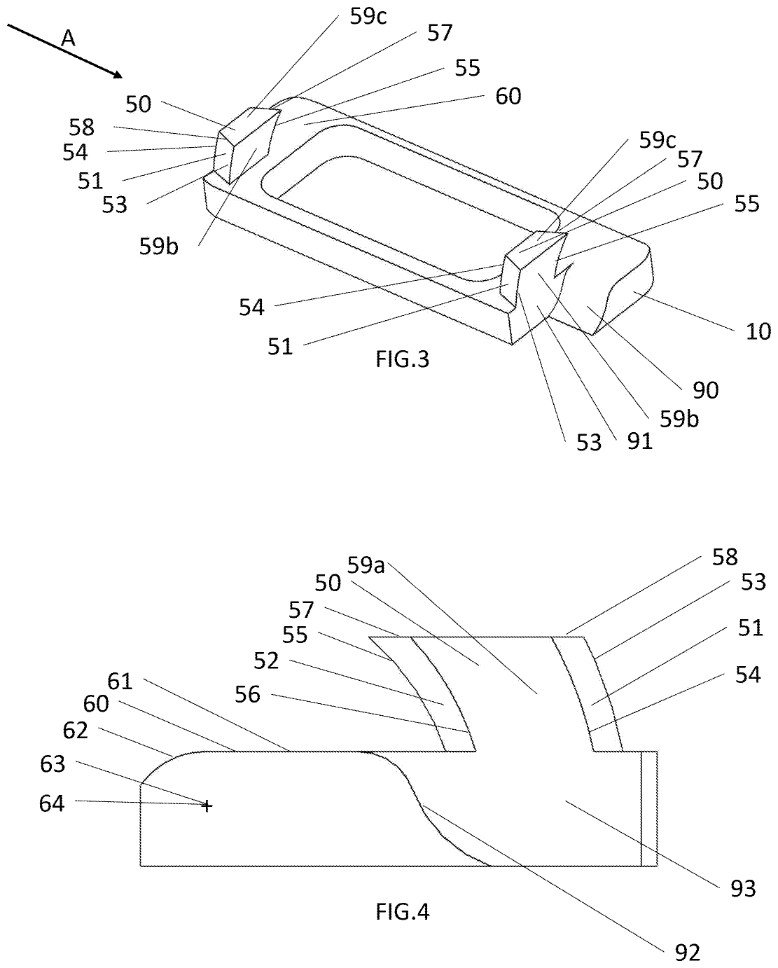

As best shown in FIGS. 3-5, the bottom component 10 has a top component facing surface 60. The top component facing surface has both a flat component 61 and a curved component 62. The two component surfaces are preferably tangent to each other, so as to ensure a smooth transition from one to another. The curved component is defined by a radius, with a center 63. The center 63 defines an axis 64 running along the length of the implant, which is the center of rotation for expansion rotations of the top component 20 with regard to the bottom component 10.

The bottom component 10 has a sheet 50 arising out of the top component facing surface 60. There is a mating slot 110 in the top component 20, generally dimensioned to receive the sheet 50. As shown in the exemplary embodiment, there are two sheets with the associated mating slots. It may be beneficial to have either fewer or more sheets and associated mating slots. As shown, both sheets are identical and both slots are identical. However, the sheets could be different, so long as the geometry defining the sheets meets the requirements described in the next two paragraphs. If the sheets are different, the matching slots would differ as well.

The sheet 50 is defined by two spaced apart surfaces 51 and 52, generally opposing faces 59a and 59b and top 59c generally aligned with upper surface 70 when assembled in the collapsed state. Surface 51 is defined by edges 53 and 54, and surface 52 is defined by edges 55 and 56. The edges 53, 54, 55 and 56 are all radii, with centers on or near axis 64. This structural relationship ensures that movements of the top component 20 relative to the bottom component 10 are permitted by both the top component facing surface 60, the one or more sheets 50 and their mating slot(s) 110. Fillets (not shown) of edge 57 would reduce the size of the top of the sheet 50 and better enable assembly, if that is of concern depending on the fit between the top component 20 and bottom component 10 with regard to the sheet 50 and mating slot 110.

The relative size of the radii 53, 54, 55 and 56 are important. As shown, the sheet 50 has a generally wedge-shaped appearance, with the leading face 59a of the sheet 50 smaller than the trailing face 59b. This is enabled by the edge radius 56 being larger than edge radius 55 and likewise edge radius 53 being larger than edge radius 54.

The bottom component 10 has two cutouts at the respective leading and trailing ends. At the leading end, the cutout is defined by a surface 93 which in this embodiment is aligned with the leading face 59a of a sheet 50, and a surface 92. Similarly, at the trailing end, the cutout is defined by a surface 91 which in this exemplary embodiment is aligned with the trailing face 59b of a sheet 50, and a surface 90.

As best shown in FIGS. 6-11, the top component 20 has a bottom component facing surface 160. The bottom component facing surface has both a flat component 161 and a curved component 162. The two component surfaces are preferably tangent to each other, so as to ensure a smooth transition from one to another. The curved component is defined by a radius, with a center 163. The center 163 defines an axis 164 running along the length of the embodiment, which is the center of rotation for expansion rotations of the top component 20 with regard to the bottom component 10. Preferably, the axis 164 is aligned with the axis 64 of the bottom component 10.

Alternatively, the bottom component facing surface 160 could just be composed of the flat component 161. Further, the bottom component facing surface could be composed of multiple surfaces enabling contact between the top and bottom components. Preferably, the bottom component facing surface 160 and the top component facing surface 60 are designed to ensure that the surfaces are in contact so as to permit load transfer between the top 20 and the bottom component 10 within the range of expansion of the embodiment.

The top component 20 has a slot 110 between the bottom component facing surface 160 and the upper surface 70. The slot 110 is generally dimensioned to receive sheet 50 of the bottom component 20. As shown, there are two slots 110. As referenced previously, it may be beneficial to have either fewer or more sheets and associated mating slots.

The slot 110 is defined by two spaced apart surfaces 151 and 152, and by generally opposing faces 159a and 159b. Surface 151 is defined by edges 153 and 154, and surface 152 is defined by edges 155 and 156. The edges 153 154 155 and 156 are all radii, with centers on or near axis 164. This ensures that movements of the top component 20 relative to the bottom component 10 are permitted by both the bottom component facing surface 160, the one or more sheets 50 and their mating slot(s) 110.

The relative size of the radii 153 154 155 and 156 are important. As shown, the slot 110 has a generally wedge-shaped appearance, with the leading face 159a of the slot 110 smaller than the trailing face 159b. This is enabled by the edge radius 156 being larger than edge radius 155 and likewise edge radius 153 being larger than edge radius 154.

The top component 20 has two extensions at the respective leading and trailing ends. At the leading end, the extension 190 is defined by the exterior surface of the top component 10, a surface 191 which in this embodiment is aligned with the leading face 159a of a slot 110 and is generally perpendicular to the axis 164, a surface 192 transitioning from the extension 190 to the bottom component facing surface 160 and a surface 193 terminating the extension 190 and generally aligned with the bottom 80 of the bottom component 10 when the embodiment is in its collapsed state. Similarly, at the trailing end, the extension 194 is defined by a surface 195 which in this embodiment is aligned with the trailing face 159b of a slot 110 and is generally perpendicular to the axis 164, a surface 196 transitioning from the extension 194 to the bottom component facing surface 160 and a surface 197 terminating the extension 194 and generally aligned with the bottom 80 of the bottom component 10 when the embodiment is in its collapsed state.

In addition, the top component 20 has a set 198 arising out of leading face 159a and surface 191. In general, the set is aligned with the locking screw 30. Further, the set has a sharp point that extends beyond the surface 191 of the extension in the direction of the cavity 40. The set could be made integral as part of the top component. Alternatively, it could be a separate component assembled into the top component 20. As a separate component, its geometry would have to be such that it could not pass through the mating hole in the top component. Geometry such as a countersink, a taper or a counter bore could be used to prevent the set from passing through the mating hole in the top component. Further, as a separate component, it might be advantageous to have a relief 199 to ease assembly of the set into the top component using a direction extending from the cavity 40 towards the extension 190.

As described above, the bottom component 10 and the top component 20 are designed to permit rotation of the top component relative to the bottom component and generally about the axes 64 and 164. The size and relative positions of the sheet(s) 50 and slot(s) 110 help ensure that this happens. The slot 110 mates with the sheet 50. However, the leading end of the slot (face 159a) must be narrower than the leading end of the sheet 59a. Likewise, the trailing end of the slot (face 159b) must be larger than the trailing end of the sheet 59b. Therefore, the slot is longer, as measured from the leading end to the trailing end, than the sheet. Further, in a position that permits generally free rotation of the top component 20 relative to the bottom component 10, the leading face 59a of the leading sheet 50 must not contact the point of the set 198.

Once the embodiment has been expanded to an appropriate position, locking the two components relative to each other is performed by tightening the locking screw 30. As the locking screw is advanced, preferably using a torque-limited driver (not shown), the leading face 31 of the locking screw 30 contacts the trailing face 59b of the trailing sheet 50 of the bottom component 10. As the screw is further advanced, the bottom component 10 translates. This translation drives the generally wedge-shaped sheet(s) 50 of the bottom component 10 towards the leading end 159a of the slot(s) 110 of the top component 20. As the locking screw 30 is further advanced, eventually the surfaces 51 and 52 of sheet 50 are jammed against surfaces 151 and 152 of slot 110, locking the two components from further movement. Simultaneously, the translation of the bottom component 20 along the direction of the locking screw 30 pushes the bottom component 20 into the set 198. Initially, the set contacts the leading faces 59a of leading sheet 50 and/or face 193 depending on the expansion angle. Further tightening of the locking screw 30 results in permanent deformation of the contact face 59a and/or 193 with the point of the set 198 of the top component 20 penetrating into the sheet 50 of the bottom component 10.

After locking, there are three different mechanisms for load transfer between the top and bottom components, resisting relative movements between them. First, by proper alignment of axes 164 and 64, there is contact between the top component facing surface 60 of the bottom component 10 and the bottom component facing surface 160 of the top component 20. Second, the jamming of the sheet(s) 50 of the bottom component 10 into the slot(s) 110 of the top component 20 creates a frictional surface that is capable of load transfer. Lastly, by interference of the set 198 of the top component 20 with the leading sheet 50 of the bottom component 10, further load transfer between the top and bottom components is enabled.

The load transfer between top and bottom component facing surfaces 60 and 160 can be adjusted, based on the relative position of the axes 64 and 164. If in the assembled, collapsed position, the axes are coincident, there will be contact throughout the expansion of the embodiment. Alternatively, if greater contact is required, the position of the axis 164 could be moved below (as shown in the drawings) axis 64. This will tend to jam the two facing surfaces 60 and 160 together as the embodiment is expanded. Contrarily, if less contact is required, the position of the axis 164 could be moved above (as shown in the drawings) axis 64, which, depending on magnitude, could result in no contact between facing surfaces 60 and 160 as the device is expanded.

Based on the previous paragraph, alternative embodiments are also considered. One alternative would eliminate the set 198, while maintaining the other features described in the embodiment described in FIGS. 1-11.

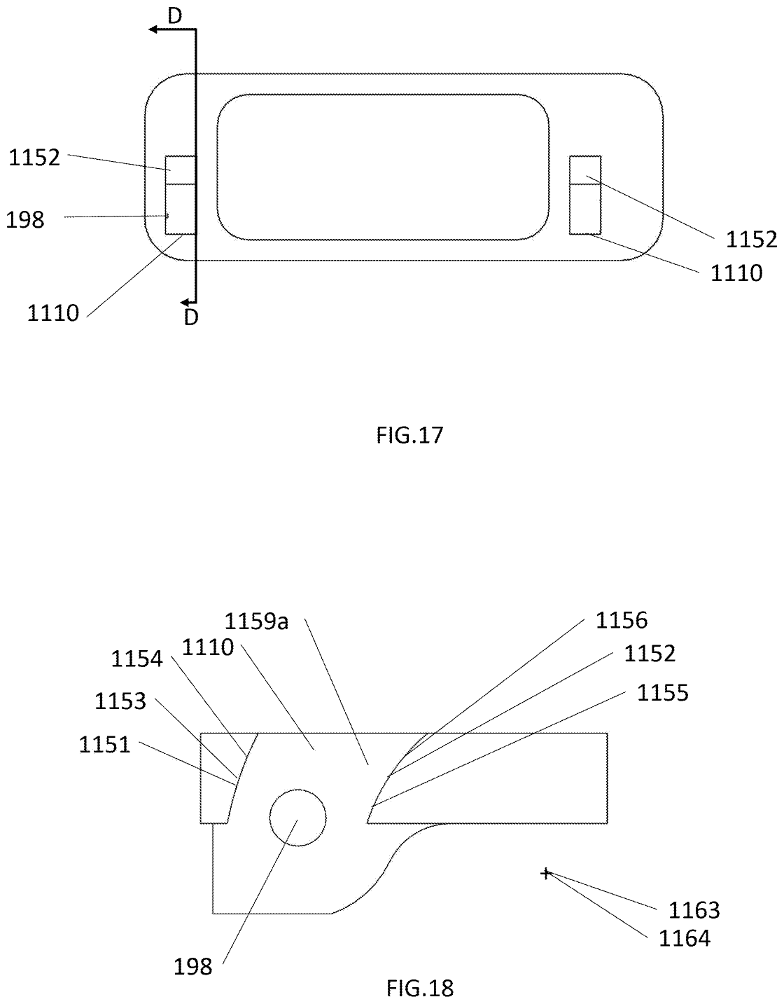

A second alternative would to replace the wedge-shape of the sheet(s) 50 and slot(s) 110 of the previous embodiment with a rectangular shape, as shown in FIGS. 12-18. In this embodiment, the mating geometry of the sheets and slots would help govern the expansion (rotation of the top component relative to the bottom component), but would not lock the two components together. Instead, locking would be provided solely by the locking screw engaging the trailing surface of the trailing sheet, and by the set engaging the leading surface of the leading sheet. Load transfer would occur through both the locking screw/set and contact between the facing surfaces.

FIG. 12 shows an oblique view of the embodiment. The embodiment is composed of a bottom component 1010, assembled with a top component 1020, with a locking screw 30 threaded into the top component. Both top and bottom components include a cavity 40 for holding bone graft materials. The outer profiles and the cavity 40 are generally matched between the top and bottom components. There is a leading end 1 and a trailing end 2 of both components. There is an upper surface 70 and a lower surface 80.

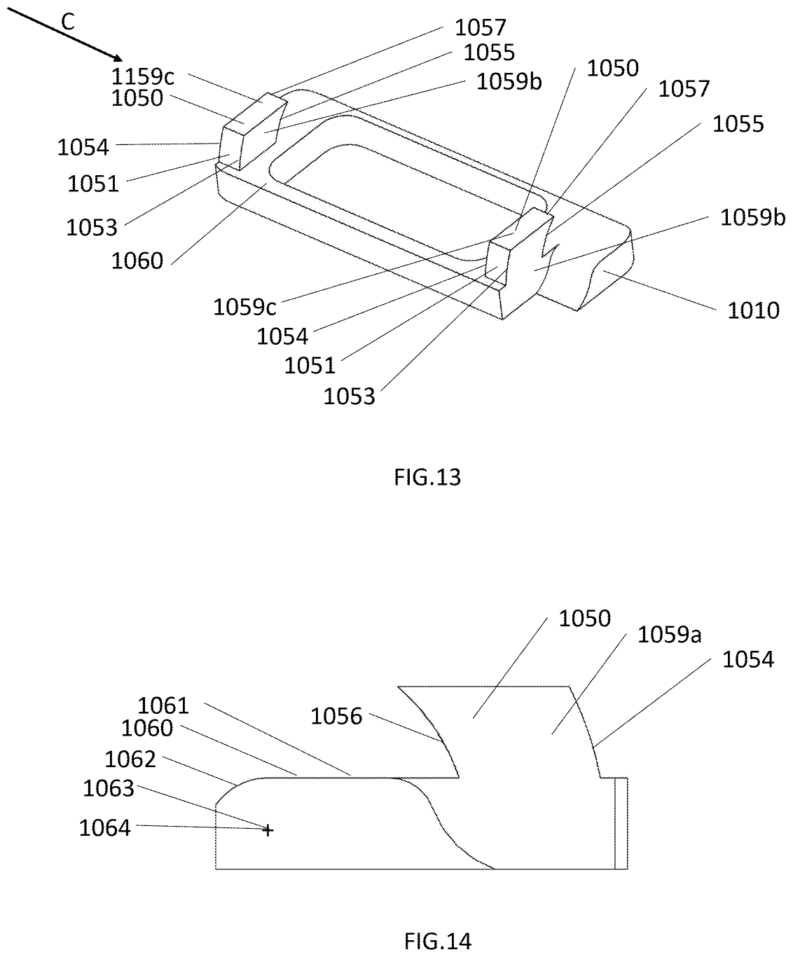

As best shown in FIGS. 13-14, the bottom component 1010 has a top component facing surface 1060. The top component facing surface has both a flat component 1061 and a curved component 1062. The two component surfaces are preferably tangent to each other, so as to ensure a smooth transition from one to another. The curved component is defined by a radius, with a center 1063. The center 1063 defines an axis 1064 running along the length of the embodiment, which is the center of rotation for expansion rotations of the top component 1020 with regard to the bottom component 1010.

The bottom component 1010 has a sheet 1050 arising out of the top component facing surface 1060. There is a mating slot 1110 in the top component 1020, generally dimensioned to receive the sheet 1050. As shown, there are two sheets with the associated mating slots. It may be beneficial to have either fewer or more sheets and associated mating slots. As shown, both sheets are identical and both slots are identical. However, the sheets could be different, so long as the geometry defining the sheets meets the requirements described in the next two paragraphs. If the sheets were different, the matching slots would differ as well.

The sheet 1050 is defined by two spaced apart surfaces 1051 and 1052, generally opposing faces 1059a and 1059b and top 1059c generally aligned with 70 when assembled in the collapsed state. Surface 1051 is defined by edges 1053 and 1054, and surface 1052 is defined by edges 1055 and 1056. The edges 1053 1054 1055 and 1056 are all radii, with centers on or near axis 64. This ensures that movements of the top component 1020 relative to the bottom component 1010 are permitted by both the top component facing surface 1060, the one or more sheets 1050 and their mating slot(s) 1110. Fillets (not shown) of edge 1057 would reduce the size of the top of the sheet 1050 and better enable assembly, if that is of concern depending on the fit between the top 1020 and bottom component 1010 with regard to the sheet 1050 and mating slot 1110.

The relative size of the radii 1053 1054 1055 and 1056 are important. As shown, sheet 1050 has a generally rectangular-shaped appearance when viewed from above. This is enabled by the edge radius 1053 equal to edge radius 1054, and likewise edge radius 1055 being equal to edge radius 1056.

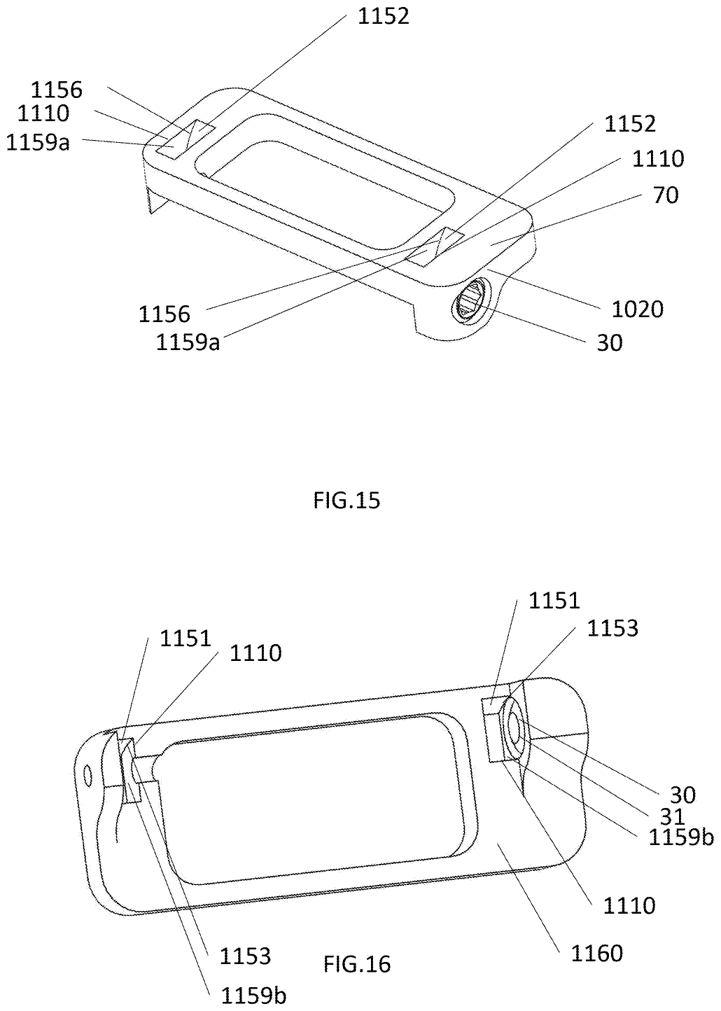

As best shown in FIGS. 15-18, the top component 1020 has a bottom component facing surface 1160. In this embodiment, the bottom component facing surface is flat.

The top component 1020 has a slot 1110 between the bottom component facing surface 1160 and the upper surface 70. The slot 1110 is generally dimensioned to receive sheet 1050 of the bottom component 1020. As shown, there are two slots 1110. As referenced previously, it may be beneficial to have either fewer or more sheets and associated mating slots.

The slot 1110 is defined by two spaced apart surfaces 1151 and 1152, and by generally opposing faces 1159a and 1159b. Surface 1151 is defined by edges 1153 and 1154, and surface 1152 is defined by edges 1155 and 1156. The edges 1153 1154 1155 and 1156 are all radii. All four radii share a common center 1163 when viewed as seen in FIG. 18. An axis 1164 runs between all center points 1163.

The relative size of the radii 1153 1154 1155 and 1156 are important. As shown, the slot 1110 has a generally rectangularly shaped appearance when viewed from above. This is enabled by the edge radius 1153 being equivalent to edge radius 1154, and likewise edge radius 1155 being equivalent to edge radius 1156.

Alignment of the axis 1164 with the axis 1064 of the bottom component 1010 when the device is in its collapsed position is preferred. This aligns the sheets 1050 with the slots 110 and enables rotation of the top component 1020 relative to the bottom component 1010 about the aligned axes 1064 and 1164. Such rotation is defined as expansion.

The present disclosure is not limited by or to the exemplary embodiments depicted/described herein, but is susceptible to revision, refinement and/or modification without departing from the spirit or scope of the present disclosure.

1. An expandable interbody implant, comprising:
a. a first component defining an upper surface;
b. a second component movable relative to the first component between a collapsed state and an expanded state,
wherein the first component includes at least one upwardly extending sheet that projects from the upper surface;
wherein the second component defines at least one mating slot configured and dimensioned to receive the at least one upwardly extending sheet of the first component;
wherein the at least one upwardly extending sheet is radiused relative to the upper surface and wherein the at least one mating slot defines an internal radius; and
wherein the radius of the at least one upwardly extending sheet and internal radius of the at least one mating slot are selected to permit relative movement between the first component and the second component when the at least one upwardly extending sheet travels relative to the at least one mating slot.

2. The expandable interbody implant of claim 1, wherein each of the first and second components defining a cavity configured and dimensioned for receipt of bone graft material;

3. The expandable interbody implant of claim 2, wherein outer profiles of the cavities defined by the first and second components are substantially matched.

4. The expandable interbody implant of claim 1, wherein the first component and the second component define abutting surfaces that are substantially parallel to each other in the collapsed state.

5. The expandable interbody implant of claim 1, wherein the first component and the second component define abutting surfaces that are angled relative to each other in the collapsed state.

6. The expandable interbody implant of claim 1, wherein the first component includes two upwardly extending sheets and wherein the second component defines two mating slots, each of the two mating slots being configured and dimensioned to receive one of the two upwardly extending sheets.

7. The expandable interbody implant of claim 1, wherein the two upwardly extending sheets are identical to each other, and wherein the two mating slots are identical to each other.

8. The expandable interbody implant of claim 1, further comprising a locking screw threadable relative to the first component.

9. A method for moving an expandable interbody implant from a collapsed orientation to an expanded orientation, comprising:
a. providing an expandable interbody implant that includes:
i. a first component defining an upper surface; and
ii. a second component movable relative to the first component between a collapsed state and an expanded state,
wherein the first component includes at least one upwardly extending sheet that projects from the upper surface;
wherein the second component defines at least one mating slot configured and dimensioned to receive the at least one upwardly extending sheet of the first component;
wherein the at least one upwardly extending sheet is radiused relative to the upper surface and wherein the at least one mating slot defines an internal radius; and
wherein the radius of the at least one upwardly extending sheet and internal radius of the at least one mating slot are selected to permit relative movement between the first component and the second component when the at least one upwardly extending sheet travels relative to the at least one mating slot; and
b. moving the first component relative to the second component between a collapsed state corresponding to a collapsed orientation and an expanded state corresponding to an expanded orientation.

10. A method according to claim 9, further comprising fixing the expandable interbody implant in an expanded orientation.
